(19) United States
(12) Patent Application Publication
(10) Pub. No.: US 2011/0112192 A1
(43) Pub. Date: May 12, 2011

(54) VERFAHREN ZUR GERUCHSEXTINKTION

(76) Inventors: Gudrun Pieper, Munster (DE); Karl-Heinz Husmann, Munster (DE)

(21) Appl. No.: 12/808,011

(22) PCT Filed: Dec. 11, 2008
(86) PCT No.: PCT/EP2008/010535

§ 371 Date: Jan. 14, 2011

(30) Foreign Application Priority Data
Dec. 13, 2007 (EP) 07024212.8

(51) Int. Cl. A61K 31/195; C07C 321/00; A61P 43/00

(52) U.S. Cl. 514/562; 562/557

(57) ABSTRACT

Use of a pharmaceutically acceptable oxidation or reduction agent for disulfide bridges containing organic compounds for the treatment of odor formation in humans due to the consumption of allium plants or allium plant stuffs.

The present invention refers to a method of the extinction of an odor and claims priority of European patent application 07024 212.8 from Dec. 13, 2007.

Allium plants are widely known as spices or side dishes in a majority of foods and preparations. This is the case, in particular with respect to garlic (allium sativum), onions (allium cepa), shallots (allium ascolonium) as well as wild garlic (allium ursinum) or chives (allium schoenoprasum). These plants each belong to the specie allium in the family of onions plants (alliaceae). Besides the pure taste component of the allium plants or their parts, which entice to consume them, health aspects are in the foreground in their consumption. It is for example known that garlic has constituents which have antimicrobial effects. In addition, many facts point to consumption of garlic leading to a lowering of fat values in the blood to thereby act preventatively against atherosclerotic changes of the blood vessels. The viscosity of the blood can also change which has been shown in in-vitro tests on human blood platelets that were treated with garlic extract.

Various medical applications are also known for onions (allium crepe). Thus, fresh juice of onions is an old home remedy against colds and coughs. In addition—similar as with garlic—there are discussions on their antimicrobial—, blood fat lowering—as well as blood coagulatory—and blood sugar lowering properties. Accordingly, the consumption of onions can have positive effects on the entire heart and circulatory system and can serve in preventing atherosclerotic changes.

A similar spectrum of effects is also assigned to chives and wild garlic. In like manner, there are reports on the inhibitory function against bacteria, the slight blood pressure lowering as well as the preventative effect of blood aggregation and a lowering of the level of cholesterol.

In summary, over centuries, a multitude of representatives of the allium plants, has been widely used due to taste but also due to their medical implication and has thus found widespread use.

Meanwhile, it is known, that the various pharmacological effects of the allium plants are due to the sulfur containing secondary plant constituents. An especially important representative of these constituents is alliin ((+)-S-allyl-L-cysteine sulfoxide). This compound, at first is odorless and probably has no pharmacological effect. It is only when the plant tissue is injured that alliin comes in contact with the alliinase and in a catalytic reaction is converted into allicin (2-propene-thiosulfinic acid (S)-allylester), which results in the typical known odor of garlic and other allium plants. Likewise, the pharmacological effect of the allium plants according to the latest level of knowledge goes back in large part to allicin, which has antibacterial, antimycotic and antibiotic effects.

Allicin is highly reactive and thus the starting product of a multitude of sulfur containing compounds that are contained in the allium plants. These are for example thioacrolein, cycloalliin or dialkylsulfides, thiosulfonate or S-oxide. Ajoen, which results from the beta elimination, together with the thioacrolein from the for example, is believed responsible for the antithrombotic effect of garlic extracts. In like manner, it causes apoptosis in human leukemia cells and has thus a potential anticancerogenic effect.

The consumption of allium plants is however accompanied with a considerable drawback in that, with the mechanical impact or breakdown of the plant tissue the allicin resulting from the catalysis of alliin forms a strong odor. This odor formation, by the overwhelming majority of people, is considered completely uncomfortable and annoying. Thus, the consumption of allium plants with its taste and medical effects is always accompanied by the drawback that persons in the nearest vicinity are suffering from this odor. However, privately as well as in the professional life, this odor should possibly be avoided.

Methods are known in the prior art that are supposed to lead to a reduced formation of odor after the consumption of allium plant material. For example, it is recommended that the annoying odor formation can be prevented during the meal preparation by washing hands with lemon after cutting onions or garlic. Alternatively, the use of “metal soap” for the break down of the odor is proposed.

These means are however effective only in the area of the hands. The compounds of allicin and others that contain disulfide bridges and produce the typical “garlic- or onion odor” after the consumption of allium containing foods, affect mostly the breath and not only the hands, so that the afore-stated means are only ineffective and inadequate.

It is thus an object of the present invention, to provide a method and an agent which reduces the odor itself or which breaks down the odor after the consumption of allium containing foods and the attendant formation of the odor.

This object is solved by applying pharmaceutically acceptable oxidation agents or reduction agents which can cleave disulfide bridges in organic molecules for treating odors in humans that are due to the consumption of allium containing plants or plant stuffs. In addition, an agent or a preparation is proposed which fulfills this purpose.

Within the scope of the present invention, the term “treatment” means every means which leads to the prevention, easing or extinction of the odor formation or the odor in or at the human body. Thus, the goal of the present invention is the intake or administration of the agent according to the present invention before, during or after the consumption of allium plants. The present invention can thus be provided preventatively as well as in lessening a progressive odor formation or an already existing odor (odor extinction).

The effect according to the present invention is essentially based on cleaving the disulfide bridges within the odor producing sulfur containing plant constituents such as for example in allicin. This cleavage can be realized either through oxidative means or through a reduction reaction.

The agents to be applied in accordance with the present invention are for example mucolytic agents. Within the scope of the present invention, these agents are defined in that they cleave disulfide bridges, especially also in organic molecules. Pharmaceutically acceptable mucolytic agents in their application are known as expectorants, that is, as mucus loosening substances. In an especially advantageous embodiment of the present invention, acetylcysteine (ACC) is used as well as its derivatives which essentially include the same biological activities as ACC. If desired, such compounds according to the present invention can also be applied.

In accordance with the present invention, pharmaceutically acceptable oxidation or reduction agents that cleave disulfide bridges are utilized.

It was already stated that the method according to the invention is the counteraction of odor formation that arises from consumption of allium plants or plant stuffs or their degradation. Within the scope of the present invention, the term “allium plants or allium plant stuffs” is to be understood as comprising any constituents of the plant source; accordingly, they include all subterranean or above ground parts inclusive of all root material, the leaves, the fruits or the seeds as well as the blossoms.

The oxidation or reduction agents can be administered or ingested in a variety of regular doses know to those skilled in the art, such as for example, as capsules, tablets, for example fizzing tablets, juice, powder or as an inhalation agent. Injection solutions are also possible. Advantageously, the dose per application of the oxidation or reduction agent (for example acetylcysteine) is below the dosing necessary for a known pharmacological effect.

The daily dose for a pharmacologic effect of the acetylcysteine as a mucal liquefier in a peroral form is at about 600 mg for adults (if desired 2-3 times daily 200 mg), with children from 6 to 14 years at 300 mg/d (or 1 to 3 times daily 100 mg), and with infants under the age of two at 2-3 times daily each 50 mg. In accordance with the present invention, the required one-time dose for adults can be at 100 mg or less per application for the observed odor extinction or prevention. Advantageously, the dose is even up to 75 mg, especially preferred up to 50 mg or up to 25 mg per application in adults. The dose can also be at about 40 mg. If required, the amount can be gained multiple times. According to experience, the dose of 0.01 mg/kg body weight up to 2.5 mg/kg, preferably from 0.05 to 2.0 mg/kg bodyweight can be administered.

The afore-stated doses can be the subject of a corresponding unit dose (for example a tablet, a sachet, a capsule or a predetermined amount of spray from an inhaler).

The compounds according to the present invention or their preparation can comprise also the usual acetylcysteine formulation adjuvants or carrier material, for example adjuvants of the group consisting of such substances as aspartame, lemon acid, sodium bicarbonate, sodium, phenyallanine or lemon aroma. This also goes for the formulation such as fizzing tablets. If the acetylcysteine is administered as a powder, it can also contain ascorbic acid (Vitamin C), saccharine, sucrose or flavorings. Additional examples of formulation contain for example, additionally lactose, mannitol, sodium in the form of sodium citrate, sodium hydrogen carbonate, sodium carbonate and sodium cyclamate. Also, saccharine-sodium and flavorings can be added.

The present invention includes also the ready for sale packaging which contain preparations of the reduction and oxidation agents according to the present invention and packaged as nutritional supplements or life-style products.

### EXAMPLES

1. Administration of acetylcysteine after the consumption of a garlic containing meal

Battery of tests with 15 tests per person.
Dosing: 50 mg with female test persons (32 to 74 kg body weight)

i. 75 mg with male test persons (52 to 98 kg body weight)

Result: no odor in all tests

In these tests, the ACC-fizzing tablets of the Hexal company were applied.

The positive test results were observed each within a time window of 10 to 20 minutes after administration of the acetylcysteine.

2. Administration of an acetylcysteine before the consumption of a garlic containing meal

Battery of tests and dosage applied the same as under 1.

The administration of acetylcysteine was done about 10 minutes before the meal. In these tests, the odor formation was likewise prevented.

1. Use of a pharmaceutically acceptable reduction agent with reactive thiol group for disulfide containing organic compounds for the production of an agent for the treatment of odor formation in humans due to the consumption of allium plant or parts thereof.

2. Use according to claim 1, characterized in that a mucolytic agent is used as reduction agent.

3. Use according to claim 1, characterized in that the agent is administered before consumption, preferably within an hour before consumption.

4. Use according to claim 1, characterized in that the agent is administered after the consumption of the allium plant.

5. Use according to claim 1, characterized in that the allium plant is one from the group of the following plants: allium sativum (garlic), allium cepa (onion), allium ursinum (wild garlic) or allium schoenoprasum (chives).

6. Use according to claim 1, characterized in that the reduction agent is acetylcysteine.

7. Use according to claim 3, characterized in that the dosage of the reduction agent is below the pharmacologically active dosage.

8. Use according to claim 7, characterized in that the dosage of acetylcysteine is between 25-125 mg, preferably between 40 and 100 mg, especially preferred between 50 and 75 mg per application.

9. Use of a pharmaceutically acceptable reduction agent for disulfide bridges containing organic compounds for the production of an agent for the treatment of odor formation in humans in a dosage unit permitting administration to a patient of 0.01 to 2.5 mg/kg bodyweight of a patient.
